(19) United States
(12) Patent Application Publication
(10) Pub. No.: US 2024/0315831 A1
(43) Pub. Date: Sep. 26, 2024

(54) BREAST IMPLANT COMPRISING SILICONE GELS OF VARIOUS PROPERTIES

(71) Applicants: Eun Jung SIM, Seoul (KR); Byung Hwi KIM, Yongin-si (KR); Min Kyoung KIM, Incheon (KR); Ju Dong SONG, Busan (KR); II Seok JANG, Yangsan-si (KR)

(72) Inventors: Eun Jung SIM, Seoul (KR); Byung Hwi KIM, Yongin-si (KR); Min Kyoung KIM, Incheon (KR); Ju Dong SONG, Busan (KR); II Seok JANG, Yangsan-si (KR)

(21) Appl. No.: 18/277,672

(22) PCT Filed: Feb. 11, 2022
(86) PCT No.: PCT/KR2022/002065

§ 371 Date: Aug. 17, 2023

(30) Foreign Application Priority Data
Feb. 19, 2021 (KR) 10-2021-0022731

(51) Int. Cl. A61F 2/12; A61L 27/18

(52) U.S. Cl. CPC A61F 2/12; A61L 27/18; A61F 2250/0014

(57) ABSTRACT

One embodiment of the present invention provides a breast implant comprising: a filler material comprising two or more types of silicone gel, having different cohesive strengths; and a silicone shell housing the filler material.

### TECHNICAL FIELD

The specification relates to a breast implant including two or more types of silicone gels having different cohesive strengths.

### BACKGROUND ART

Silicone gel breast implants are manufactured by filling a silicone gel having appropriate viscosity in a silicone shell forming the outer shell of the implant and have excellent durability and tactile sensation compared to saline breast implants. The silicone gel breast implant is a Class IV medical device and absolutely requires special biocompatibility, safety, and stability because it is generally present for a long period of time after being implanted in vivo. Also, in the case of a breast implant, a natural shape or a texture similar to skin tissue need to be realized. Therefore, it has been developed to secure properties required as a medical device by adjusting the thickness of a silicone shell or changing a filler component and simultaneously have a natural shape, but there are other side effects on the shape

A rippling phenomenon (rippling deformity) refers to a folding phenomenon of an implant shell and means a phenomenon in which wavy wrinkles are formed on an implant and revealed through the skin. This phenomenon is one of the side effects that frequently occur in use of silicone gel breast implants and may lead to a reoperation. The major cause of rippling is the downward tilting of a gel, and when the implant is present in an upright state for a long period of time after being implanted in vivo, the silicone gel filled in the implant tilts downward due to gravity, and thus wrinkles are formed in the area filled with a relatively small amount of silicone gel. Therefore, the rippling phenomenon continuously occurs at a specific site of the implant, and the properties of the shell folded at the corresponding site are degraded, and thus the implant may subsequently rupture in vivo due to an impact.

When the silicone gel breast implant ruptures, the silicone gel filled in the implant may leak out. The long-term leakage of the silicone gel may change the volume and shape of the implant, and the leaked silicone gel may adversely affect internal tissues or organs, leading to severe side effects such as complications, tissue necrosis, and the like.

### DISCLOSURE

### Technical Problem

The specification is designed to solve the above-described problems of the related art, and the present invention is directed to providing a silicone gel breast implant having excellent stability by including two or more types of silicone gels having different cohesive strengths.

### Technical Solution

One aspect of the present invention provides a breast implant which includes: a filler including two or more types of silicone gels having different cohesive strengths; and a silicone shell surrounding the filler.

In an embodiment, an inside of the silicone shell may be divided into two or more areas, and the two or more types of silicone gels having different cohesive strengths may be disposed in the two or more areas, respectively.

In an embodiment, the inside of the silicone shell may be divided into a side portion and a middle portion, and two types of silicone gels having different cohesive strengths may be disposed in the side portion and the middle portion, respectively.

In an embodiment, the silicone gel disposed in the side portion may have higher cohesive strength than the silicone gel disposed in the middle portion.

In an embodiment, the silicone gel disposed in the side portion may have a cohesive strength of 10 to 40 N.

In an embodiment, the side portion may occupy 10 to 50 vol % based on a total volume of the inside of the silicon shell.

In an embodiment, the inside of the silicone shell may be divided into an upper portion and a lower portion, and two types of silicone gels having different cohesive strengths may be disposed in the upper portion and the lower portion, respectively.

In an embodiment, the silicone gel disposed in the lower portion may have higher cohesive strength than the silicone gel disposed in the upper portion.

In an embodiment, the silicone gel disposed in the lower portion may have a cohesive strength of 10 to 40 N.

In an embodiment, the lower portion may occupy 10 to 50 vol % based on the total volume of the inside of the silicon shell.

### Advantageous Effects

Since a breast implant according to an aspect of the specification includes two or more types of silicone gels having different cohesive strengths, a rippling phenomenon, which is a side effect that occurs in a silicone gel breast implant, is prevented, and thus the risk of in vivo rupture can be reduced.

However, it is to be understood that the effect of an aspect of the specification is not limited to the above-described effect but include all effects deducible from the configuration described in the detailed description of the specification or in the claims.

### DESCRIPTION OF DRAWINGS

FIG. 1 shows a longitudinal sectional view of a breast implant having reinforced gel cohesion in a side portion according to an embodiment of the specification.

FIG. 2 shows a transverse sectional view of a breast implant having reinforced gel cohesion in a side portion according to an embodiment of the specification.

FIG. 3 shows a longitudinal sectional view of a breast implant having reinforced gel cohesion in a lower portion according to an embodiment of the specification.

FIG. 4 shows a transverse sectional view of a lower portion of a breast implant having reinforced gel cohesion in a lower portion according to an embodiment of the specification.

### MODES OF THE INVENTION

Hereinafter, an aspect of the specification will be described with reference to the accompanying drawings. However, it should be understood that the descriptions of the specification can be implemented in various other forms, and that it is not intended to limit the present invention to the exemplary embodiments. Also, in the drawings, descriptions of parts unrelated to the detailed description are omitted to clearly describe an aspect of the specification. Throughout the specification, like numbers refer to like elements.

Throughout the specification, the phrase a certain part is “connected” to another part means that the certain part is not only “directly connected” to the other part but also “indirectly connected” to the other part through another member interposed between the two parts. Also, the phrase a certain part “includes” a certain element means that the certain part may further include, instead of excluding, another element unless particularly indicated otherwise.

When a numerical value is presented in the specification, the value has the precision of significant digits provided in accordance with the standard rules in chemistry for significant digits unless its specific range is stated otherwise. For example, the numerical value 10 includes the range of 5.0 to 14.9 and the numerical value 10.0 includes the range of 9.50 to 10.49.

Hereinafter, an embodiment of the specification will be described in detail with reference to the accompanying drawings.

### Breast Implant Including Silicone Gels Having Different Cohesive Strengths

A breast implant according to an aspect of the specification includes: a filler including two or more types of silicone gels having different cohesive strengths; and a silicone shell surrounding the filler.

The cross-section of the breast implant may have one shape selected from a circular shape, an oval shape, and a teardrop shape, but the present invention is not limited thereto. Generally, since users have different breast shapes, the type of the breast implant may be selected in consideration of an original shape and preference of users such as a desired shape, a desired size, and the like.

The silicone gel may be polyorganosiloxane. The polyorganosiloxane may be one or more selected from the group consisting of dimethylsiloxane, methyl hydrogen siloxane, methylphenylsiloxane, diphenylsiloxane, dimethylvinylsiloxane, and trifluoropropylsiloxane, but the type thereof is not particularly limited as long as it can realize the functional characteristics of a breast implant.

The two or more types of silicone gels may have different properties such as viscosity, elasticity, flowability, and the like.

Since the breast implant according to an aspect of the specification includes two or more types of silicone gels having different cohesive strengths, the degree at which the gel tilts downward can be adjusted, the resistance of a site where folding occurs in the implant can be increased to prevent a rippling phenomenon, and the stability of the implant can be enhanced. In the case of a conventional breast implant including a silicone gel having a single property, a rippling phenomenon frequently occurs due to the downward tilting of a gel and becomes a major cause of implant rupture.

The silicone shell may be manufactured by applying a silicone polymer dispersion onto a mandrel having a desired shape and performing curing. The silicone polymer dispersion may form an elastomer coating on the mandrel, and the coating may be cured, and the solvent may be evaporated.

An inside of the silicone shell may be divided into two or more areas, and the two or more types of silicone gels having different cohesive strengths may be disposed in the two or more areas, respectively. For example, the two or more areas may be divided by an interface formed between the two or more types of silicone gels having different cohesive strengths.

The inside of the silicone shell may be divided into an area where a rippling phenomenon occurs and an area where a rippling phenomenon does not occur, and the silicone gel disposed in the area where a rippling phenomenon occurs may have higher cohesive strength, higher viscosity, or lower flowability than the silicone gel disposed in the area where a rippling phenomenon does not occur.

Since a silicone gel having high cohesive strength, high viscosity, or low flowability is disposed in the area where a rippling phenomenon occurs in the inside of the silicone shell, the breast implant according to an aspect of the specification can prevent a rippling phenomenon and increase rupture resistance to enhance the stability of the implant.

### Breast Implant Having Reinforced Gel Cohesion in Side Portion

In the breast implant according to an aspect of the specification, the inside of the silicone shell may be divided into a side portion 12 and a middle portion 13, and two types of silicone gels having different cohesive strengths may be disposed in the side portion 12 and the middle portion 13, respectively.

The side portion 12 may be formed along a side circumference of the breast implant, and the middle portion 13 may refer to all areas except for the side portion 12 in the inside of the silicone shell.

The silicone gel disposed in the side portion 12 may have higher cohesive strength than the silicone gel disposed in the middle portion 13.

The silicone gel disposed in the side portion 12 may have higher viscosity and lower flowability than the silicone gel disposed in the middle portion 13.

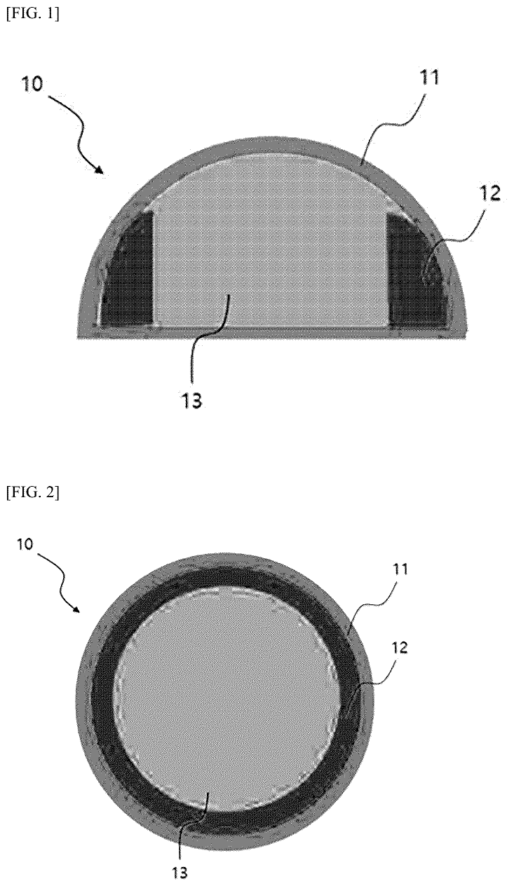

FIGS. 1 and 2 show the longitudinal sectional view and transverse sectional view of a breast implant having reinforced gel cohesion in a side portion according to an embodiment of the specification, respectively.

Referring to FIGS. 1 and 2, a breast implant 10 having reinforced gel cohesion in a side portion according to an embodiment of the specification may include: a silicone shell 11; a side portion 12 filled with a cohesive strength-reinforced silicone gel; and a middle portion 13 filled with a general silicone gel.

Since the side portion 12 is filled with a cohesive strength-reinforced silicone gel having high cohesive strength compared to the middle portion 13, the degree at which the gel tilts downward can be adjusted, the resistance of a site where folding occurs in the implant can be increased to prevent a rippling phenomenon, and the stability of the implant can be enhanced.

Since the middle portion 13 is filled with a general silicone gel having lower cohesive strength than the cohesive strength-reinforced silicone gel filled in the side portion 12, the breast implant can realize a natural shape and a texture similar to skin tissue.

The silicone gel disposed in the side portion 12 may have a cohesive strength of 10 to 40 N. For example, the silicone gel disposed in the side portion 12 may have a cohesive strength of 10 N, 11 N, 12 N, 13 N, 14 N, 15 N, 16 N, 17 N, 18 N, 19 N, 20 N, 21 N, 22 N, 23 N, 24 N, 25 N, 26 N, 27 N, 28 N, 29 N, 30 N, 31 N, 32 N, 33 N, 34 N, 35 N, 36 N, 37 N, 38 N, 39 N, 40 N, or a cohesive strength in a range between two of the above values. When the silicone gel disposed in the side portion 12 has a cohesive strength less than 10 N, a rippling phenomenon occurs, and thus the possibility of implant rupture may increase, and when the silicone gel disposed in the side portion 12 has a cohesive strength exceeding 40 N, a natural shape or a texture similar to skin tissue may not be realized.

The side portion 12 may occupy 10 to 50 vol % based on the total volume of the inside of the silicone shell 11. For example, the volume of the side portion 12 may be 10 vol %, 15 vol %, 20 vol %, 25 vol %, 30 vol %, 35 vol %, 40 vol %, 45 vol %, 50 vol %, or in a range between two of the above values based on the total volume of the inside of the silicone shell 11. When the volume of the side portion 12 is less than 10 vol % based on the total volume of the inside of the silicone shell 11, a rippling phenomenon occurs, and thus the possibility of implant rupture may increase, and when the volume of the side portion 12 exceeds 50 vol %, a natural shape or a texture similar to skin tissue may not be realized.

### Breast Implant Having Reinforced Gel Cohesion in Lower Portion

In the breast implant according to an aspect of the specification, the inside of the silicone shell may be divided into an upper portion 23 and a lower portion 22, and two types of silicone gels having different cohesive strengths may be disposed in the upper portion 23 and the lower portion 22, respectively.

The upper portion 23 may refer to an area located at the front when the breast implant is implanted in vivo, and the lower portion 22 may refer to an area located at the rear when the breast implant is implanted in vivo.

The silicone gel disposed in the lower portion 22 may have higher cohesive strength than the silicone gel disposed in the upper portion 23.

The silicone gel disposed in the lower portion 22 may have higher viscosity and lower flowability than the silicone gel disposed in the upper portion 23.

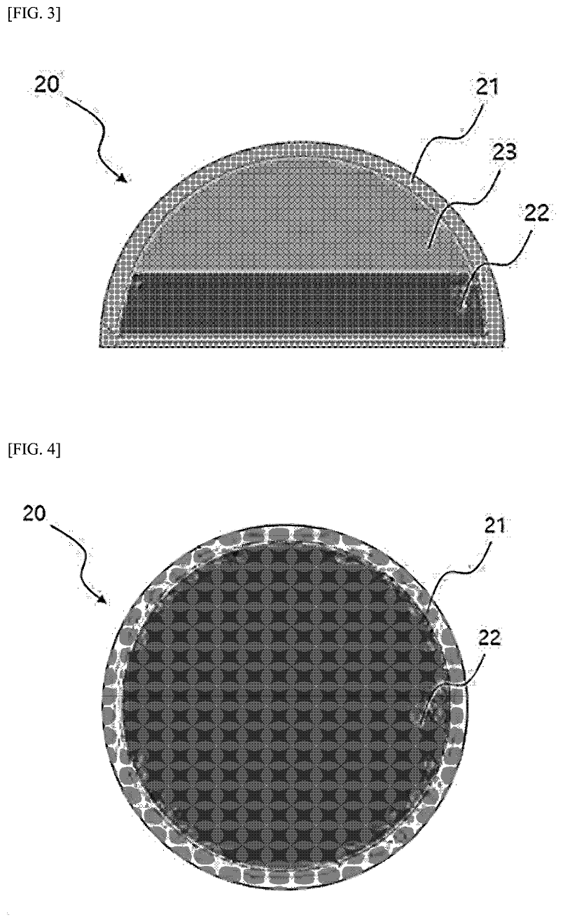

FIGS. 3 and 4 show the longitudinal sectional view of a breast implant having reinforced gel cohesion in a lower portion according to an embodiment of the specification and the transverse sectional view of the lower portion of the breast implant, respectively.

Referring to FIGS. 3 and 4, a breast implant 20 having reinforced gel cohesion in a lower portion according to an embodiment of the specification may include: a silicone shell 21; a lower portion 22 filled with a cohesive strength-reinforced silicone gel; and an upper portion 23 filled with a general silicone gel.

Since the lower portion 22 is filled with a cohesive strength-reinforced silicone gel having high cohesive strength compared to the upper portion 23, the degree at which the gel tilts downward can be adjusted, the resistance of a site where folding occurs in the implant can be increased to prevent a rippling phenomenon, and the stability of the implant can be enhanced.

Since the upper portion 23 is filled with a general silicone gel having lower cohesive strength than the cohesive strength-reinforced silicone gel filled in the lower portion 22, the breast implant can realize a natural shape and a texture similar to skin tissue.

The silicone gel disposed in the lower portion 22 may have a cohesive strength of 10 to 40 N. For example, the silicone gel disposed in the lower portion 22 may have a cohesive strength of 10 N, 11 N, 12 N, 13 N, 14 N, 15 N, 16 N, 17 N, 18 N, 19 N, 20 N, 21 N, 22 N, 23 N, 24 N, 25 N, 26 N, 27 N, 28 N, 29 N, 30 N, 31 N, 32 N, 33 N, 34 N, 35 N, 36 N, 37 N, 38 N, 39 N, 40 N, or a cohesive strength in a range between two of the above values. When the silicone gel disposed in the lower portion 22 has a cohesive strength less than 10 N, a rippling phenomenon occurs, and thus the possibility of implant rupture may increase, and when the silicone gel disposed in the lower portion 22 has a cohesive strength exceeding 40 N, a natural shape or a texture similar to skin tissue may not be realized.

The lower portion 22 may occupy 10 to 50 vol % based on the total volume of the inside of the silicone shell 21. For example, the volume of the lower portion 22 may be 10 vol %, 15 vol %, 20 vol %, 25 vol %, 30 vol %, 35 vol %, 40 vol %, 45 vol %, 50 vol %, or in a range between two of the above values based on the total volume of the inside of the silicone shell 21. When the volume of the lower portion 22 is less than 10 vol % based on the total volume of the inside of the silicone shell 21, a rippling phenomenon occurs, and thus the possibility of implant rupture may increase, and when the volume of the lower portion 22 exceeds 50 vol %, a natural shape or a texture similar to skin tissue may not be realized.

Hereinafter, examples of the specification will be described in further detail. However, the following experimental results are obtained from only a few selected examples of the invention, and the scope and contents of the specification should not be interpreted as being reduced or limited by the few selected examples. The effects of each of the various embodiments of the specification, which are not explicitly set forth below, will be described in detail in relevant sections.

### Example 1: Breast Implant Having Reinforced Gel Cohesion in Side Portion

A side portion 12 occupying 20 vol % based on the total volume of an inside of a silicone shell 11 was filled with a cohesive strength-reinforced silicone gel having a cohesive strength of 23 N, and a middle portion 13 was filled with a general silicone gel having a cohesive strength of 13 N, thereby manufacturing a breast implant 10 having reinforced gel cohesion in the side portion.

### Example 2: Breast Implant Having Reinforced Gel Cohesion in Lower Portion

An upper portion 23 was filled with a general silicone gel having a cohesive strength of 13 N, and a lower portion 22 occupying 30 vol % based on the total volume of an inside of a silicone shell 21 was filled with a cohesive strength-reinforced silicone gel having a cohesive strength of 26 N, thereby manufacturing a breast implant 20 having reinforced gel cohesion in the lower portion.

### Comparative Example 1

A breast implant 10 having reinforced gel cohesion in a side portion was manufactured in the same manner as in Example 1, except that the cohesive strength-reinforced silicone gel filled in a side portion 12 had a cohesive strength of 15 N.

### Comparative Example 2

A breast implant 10 having reinforced gel cohesion in a side portion was manufactured in the same manner as in Example 1, except that the volume of a side portion 12 occupied 5 vol % based on the total volume of an inside of a silicone shell 11.

### Comparative Example 3

A breast implant 20 having reinforced gel cohesion in a lower portion was manufactured in the same manner as in Example 2, except that the cohesive strength-reinforced silicone gel filled in a lower portion 22 had a cohesive strength of 15 N.

### Comparative Example 4

A breast implant 20 having reinforced gel cohesion in a lower portion was manufactured in the same manner as in Example 2, except that the volume of a lower portion 22 occupied 10 vol % based on the total volume of an inside of a silicone shell 21.

### Comparative Example 5: Breast Implant Including Silicone Gel Having Single Property

A general silicone gel having a cohesive strength of 13 N was filled in the entire inside of the silicone shell to manufacture a breast implant including a silicone gel having a single property.

The cohesive strength of each gel in the examples and comparative examples is a value obtained by measuring the pressure at the time the silicone gel discharged from a 1-mm circular hole is separated when negative pressure is applied after the hole is made in the outside of the implant.

### Experimental Example 1: Observation of Shape Change

In order to confirm the effect of the breast implant including silicone gels having various properties according to an aspect of the specification on prevention of a rippling phenomenon, a change in shape of each implant according to Examples 1 and 2 and Comparative Examples 1 to 5 when one side of the implant was held and lifted was observed.

As a result, in the case of the implant of Comparative Example 2 in which the volume filled with a cohesive strength-reinforced silicone gel occupied less than 10 vol % based on the total volume of the inside of the silicone shell 21 and the implant of Comparative Example 5 in which a silicone gel filled therein had a single property, the downward tilting of the gel due to gravity and the rippling phenomenon were observed. On the other hand, in the case of the implants of Examples 1 and 2, the downward tilting of the gel due to gravity was significantly reduced, and rippling did not occur, as compared with the implants of Comparative Examples 1 to 5.

### Experimental Example 2: Evaluation of Rupture Safety and Stability

In order to confirm the effect of the breast implant including silicone gels having various properties according to an aspect of the specification on reduction in the risk of rupture, fatigue was applied 6 million times to each implant of Examples 1 and 2 and Comparative Examples 1 to 5, and rupture safety and stability were evaluated.

Referring Table 1 below, in the case of the implant of Comparative Example 2 in which the volume filled with a cohesive strength-reinforced silicone gel occupied less than 10 vol % based on the total volume of the inside of the silicone shell 21 and the implant of Comparative Example 5 in which a silicone gel filled therein had a single property, when fatigue was applied 4 million times or more, the side portion ruptured, and the internal filler leaked. On the other hand, in the case of the implants of Examples 1 and 2, it was confirmed that, even when fatigue was applied 6 million times or more, rupture did not occur, and thus rupture safety and stability were excellent.

In the case of the implants of Comparative Examples 1 and 3 using a cohesive strength-reinforced silicone gel having a cohesive strength of 15 N and the implant of Comparative Example 4 in which the volume filled with a cohesive strength-reinforced silicone gel occupied 10 vol % based on the total volume of the inside of the silicone shell, when fatigue was applied 6 million times or more, rupture occurred. From this result, it was confirmed that rupture safety and stability were improved compared to the implants of Comparative Examples 2 and 5.

TABLE 1

| Classification | 2 million times | 4 million times | 6 million times |
|---|---|---|---|
| Example 1 | Nothing wrong | Nothing wrong | Nothing wrong |
| Example 2 | Nothing wrong | Nothing wrong | Nothing wrong |
| Comparative | Nothing wrong | Nothing wrong | Rupture of |
| Example 1 | | | side portion |
| Comparative | Nothing wrong | Rupture of | Rupture of |
| Example 2 | | side portion | side portion |
| Comparative | Nothing wrong | Nothing wrong | Rupture of |
| Example 3 | | | side portion |
| Comparative | Nothing wrong | Nothing wrong | Rupture of |
| Example 4 | | | side portion |
| Comparative | Nothing wrong | Rupture of | Rupture of |
| Example 5 | | side portion | side portion |

The foregoing description of the specification is intended for illustration, and it will be understood by those skilled in the art to which the invention pertains that the invention can be easily modified in other specific forms without changing the technical spirit or essential features described in the specification. Therefore, it should be understood that the examples described above are only exemplary in all aspects and not limiting. For example, each of the constituents described as being one combined entity may be implemented separately, and similarly, constituents described as being separate entities may be implemented in a combined form.

It should be understood that the scope of the specification is defined by the following claims and that all changes or modifications derived from the meaning and scope of the claims and their equivalents are included in the scope of the specification.

### LIST OF REFERENCE NUMERALS

10: Breast implant having reinforced gel cohesion in side portion
11: Silicone shell
12: Side portion
13: Middle portion
20: Breast implant having reinforced gel cohesion in lower portion
21: Silicone shell
22: Lower portion
23: Upper portion

1. A breast implant comprising:
a filler including two or more types of silicone gels having different cohesive strengths; and
a silicone shell surrounding the filler.

2. The breast implant of claim 1, wherein an inside of the silicone shell is divided into two or more areas, and the two or more types of silicone gels having different cohesive strengths are disposed in the two or more areas, respectively.

3. The breast implant of claim 1, wherein the inside of the silicone shell is divided into a side portion and a middle portion, and two types of silicone gels having different cohesive strengths are disposed in the side portion and the middle portion, respectively.

4. The breast implant of claim 3, wherein the silicone gel disposed in the side portion has higher cohesive strength than the silicone gel disposed in the middle portion.

5. The breast implant of claim 3, wherein the silicone gel disposed in the side portion has a cohesive strength of 10 to 40 N.

6. The breast implant of claim 3, wherein the side portion occupies 10 to 50 vol % based on a total volume of the inside of the silicon shell.

7. The breast implant of claim 1, wherein the inside of the silicone shell is divided into an upper portion and a lower portion, and two types of silicone gels having different cohesive strengths are disposed in the upper portion and the lower portion, respectively.

8. The breast implant of claim 7, wherein the silicone gel disposed in the lower portion has higher cohesive strength than the silicone gel disposed in the upper portion.

9. The breast implant of claim 7, wherein the silicone gel disposed in the lower portion has a cohesive strength of 10 to 40 N.

10. The breast implant of claim 7, wherein the lower portion occupies 10 to 50 vol % based on the total volume of the inside of the silicon shell.
